(19) United States
(12) Patent Application Publication
(10) Pub. No.: US 2025/0339479 A1
(43) Pub. Date: Nov. 6, 2025

(54) PROBIOTIC BLEND SUPPLEMENT WITH MICRONUTRIENTS FOR INFANT AND CHILDREN'S HEALTH

(71) Applicant: ResBiotic Nutrition, Inc., Birmingham, AL (US)

(72) Inventor: Charitharth Vivek Lal, Vestavia Hills, AL (US)

(73) Assignee: ResBiotic Nutrition, Inc., Birmingham, AL (US)

(21) Appl. No.: 18/995,589

(22) PCT Filed: Jul. 26, 2023
(86) PCT No.: PCT/US2023/071004

§ 371 Date: Jan. 16, 2025

Related U.S. Application Data
(60) Provisional application No. 63/369,716, filed on Jul. 28, 2022.

(51) Int. Cl. A61K 35/747; A61K 35/745

(52) U.S. Cl. CPC A61K 35/747; A61K 35/745

(57) ABSTRACT

The present disclosure relates to a probiotic composition that delivers a mixture or blend of 7 probiotic strains including Lactobacillus spp. (L. plantarum, L. acidophilus, and L. rhamnosus) and Bifidobacterium spp. (B. lactis, B. infantis, B. breve, and B. longum). The probiotic compositions of the disclosure comprise these 7 strains that may either be a live organism capable of proliferation in the subject, a non-living strain such as, but not limited to, a heat-killed proliferative strain, or a combination thereof. In addition to the Lactobacillus and Bifidobacterium strains, the compositions can include micronutrients such as Vitamin D, zinc, lactoferrin, HMOs, DHA+ARA, and inulin, in various combinations to support infant health.

### TECHNICAL FIELD

This invention relates a probiotic blend of 7 probiotic strains including Lactobacillus spp. (L. plantarum, L. acidophilus, and L. rhamnosus) and Bifidobacterium spp. (B. lactis, B. infantis, B. breve, and B. longum). The probiotic compositions of the disclosure comprise these 7 strains that may either be a live organism capable of proliferation in the subject, a non-living strain such as, but not limited to, a heat-killed proliferative strain, or a combination thereof. In addition to the Lactobacillus and Bifidobacterium strains, the compositions can include micronutrients such as Vitamin D, zinc, lactoferrin, HMOs, DHA+ARA, and inulin, in various combinations to support infant health.

### BACKGROUND

Children and infants require critical nutrients to support their health throughout their growth and development. Preterm infants are at high risk for lung and gut dysbiosis and micronutrient deficiencies during the critical postnatal growth period. Infants and children are also susceptible, and early exposure to noxious stimuli, supplemental oxygen, or infection can have long-lasting effects on pulmonary and intestinal health well into adulthood. Supplementation with commensal bacteria strains and key micronutrients may support infant and children's health and reduce the risk of developing comorbidities of the gut and lung.

Certain nutrients are particularly relevant to early childhood development at different stages. Vitamin D3, lactoferrin, and zinc have been studied for their use in infants and children, supporting healthy lungs, gut, and growth. Children have also shown benefit from DHA and ARA for brain health and development and prebiotic fiber for healthy digestion.

If a way to provide a blend of commensal Lactobacillus and Bifidobacterium strains could be found in order to support early infant health and development, this would be a contribution to the art. Additional formulations may include DHA+ARA and prebiotic fiber, or other vitamins, or other micronutrients.

### SUMMARY

In one embodiment a dietary supplement, food product, or nutraceutical is described for use in supporting health in a human infant or child, comprising a blend of bacterial strains selected from the group consisting of Lactobacillus spp. including L. plantarum, L. acidophilus, and L. rhamnosus, and Bifidobacterium spp. including B. lactis, B. infantis, B. breve, and B. longum. The supplements herein may be formulated for oral administration.

Methods for improving gastrointestinal or respiratory health in an infant or child are described, including providing a dietary supplement including a blend of bacterial strains selected from the group consisting of Lactobacillus spp. including L. plantarum, L. acidophilus, and L. rhamnosus, and Bifidobacterium spp. including B. lactis, B. infantis, B. breve, and B. longum; and administering the blend to the infant or child by oral administration.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 depicts growth of combined Bifidobacterium strains: showing B. lactis (RSB14™), B. infantis (RSB15™M), B. breve (RSB16™), and B. longum (RSB17™) are not inhibited by lactoferrin, Vitamin D3, or zinc gluconate when treated with those test compounds at 0.15 mg/mL, 1.5 mg/mL, and 15 mg/mL, respectively.

FIG. 2 depicts growth of combined Lactobacillus strains: showing L. plantarum (RSB11™), L. acidophilus (RSB12™), and L. rhamnosus (RSB13™) are not inhibited by lactoferrin or zinc gluconate when treated with those test compounds at 0.15 mg/mL, 1.5 mg/mL, and 15 mg/mL, respectively.

FIG. 3 depicts growth of Lactobacillus strains: showing L. plantarum (RSB11™), L. acidophilus (RSB12™), and L. rhamnosus (RSB13™) are not inhibited as individual strains by Vitamin D3 when treated with Vitamin D3 at 10 IU/mL, 100 IU/mL, 1,000 IU/mL, 10,000 IU/mL, and 100,000 IU/mL, respectively.

FIG. 4 shows that treatment of human bronchial epithelial (HBE) cells exposed to E. coli with Vitamin D3 at 30 IU/mL, 60 IU/mL, 30 IU/mL, and 30 IU/mL reduced MMP-9 mRNA levels.

FIG. 5 shows that treatment of human bronchial epithelial (HBE) cells exposed to E. coli with B. lactis (RSB14™), B. infantis (RSB15™), B. breve (RSB16™), and B. longum (RSB17™) strains, each individually, and all combined as a blend (“BF”), respectively, reduced MMP-9 mRNA levels.

### DETAILED DESCRIPTION

The present disclosure encompasses embodiments of a probiotic composition that delivers resB blend 2, which comprises a mixture of 7 probiotic strains including Lactobacillus (L. plantarum (RSB11™M), L. acidophilus (RSB12™M), and L. rhamnosus (RSB13™)) and Bifidobacterium (B. lactis (RSB14™), B. infantis (RSB15™″), B. breve (RSB16™), and B. longum (RSB17™)). The probiotic compositions of the disclosure comprise these 7 strains that may either be a live organism capable of proliferation in the subject, a non-living strain such as, but not limited to, a heat-killed proliferative strain, or a combination thereof. In addition to the Lactobacillus and Bifidobacterium strains, the compositions can include micronutrients such as Vitamin D, lactoferrin, and zinc to support human infant or children's health. These micronutrients include nutritionally active ingredients.

Each of the above bacterial strains, either alone or in combination, are available from ResBiotic Nutrition, Inc. (Birmingham, Alabama) as isolated and viable strains.

In one embodiment, the seven bacterial strains cited above may be included in combination as a blend, for example, as resB blend 2.

In another embodiment, a “bacterial extract” of one or more of the seven bacterial strains may be included in the blend. Generally, bacterial extracts are cellular components of the bacteria including, but not limited to, cell supernatant, exosomes, and cell wall material or are metabolic byproducts of bacteria such as lactic acid.

Other useful bacterial strains include, but are not limited to, L. plantarum Lp-202195, L. acidophilus NCFM, L. rhamnosus GG, L. rhamnosus HN001, B. lactis HN019, B. lactis DSM 15954, and B. lactis Bi-07. Any of the aforementioned strains may be used in the blends described herein.

The Lactobacillus genus is extremely diverse and expanding every year. With over

230 species, it has grown into one of the biggest genera in the bacterial taxonomy. As the genus has exceeded the acceptable “normal diversity,” renaming and re-classification is inevitable wherein the genus Lactobacillus may be split into most likely twelve new genera. Many traditional “probiotic” species with substantiated industrial importance and starter cultures many no longer eventually be called “Lactobacillus.” Hence, a substantial communication challenge looms ahead to reduce the inevitable confusion regarding the “old commercial” and “correct scientific” nomenclature. Once the International Committee on Systematics of Prokaryotes publishes new nomenclature in their official journal, the INTERNATIONAL JOURNAL OF SYSTEMATIC AND EVOLUTIONARY MICROBIOLOGY, the changes are valid and official. The manuscript that will be submitted for publication outlining the new nomenclature of the Lactobacillus genus will likely be ready for submission by the end of 2018. Meanwhile, there was a taxonomic subcommittee meeting in September 2018 to discuss the nomenclature changes and an (invite-only) expert LABIP workshop in October 2018 that will evaluate the science while considering the consequences for regulations, legal/IP, and industry.

Probiotics are measured by colony forming units (“CFUs”). Few studies have been done to determine effective dosages, but effective dosages are usually in the hundreds of millions of CFUs or higher. If probiotics are being used to help with digestion, probiotics should be taken with meals, but otherwise the probiotics may survive better if taken between meals, particularly if taken with liquids that help to dilute stomach acid and move the probiotics more quickly into the digestive tract. Probiotics may be given short-term (e.g., in a daily dose for days or weeks) or long-term (over several months, or more).

In some implementations, the concentration of the probiotic microorganism in the composition may be at least about 1·10⁹ CFU/g, at least about 2·10⁹ CFU/g, at least about 3·10⁹ CFU/g, at least about 4·10⁹ CFU/g, at least about 5·10⁹ CFU/g, at least about 6·10⁹ CFU/g, at least about 7·10⁹ CFU/g, at least about 8·10⁹ CFU/g, at least about 9·10⁹ CFU/g, at least about 1·10¹⁰ CFU/g, at least about 2·10¹⁰ CFU/g, at least about 3 ·10¹⁰ CFU/g, at least about 4·10¹⁰ CFU/g, at least about 5·10¹⁰ CFU/g, at least about 6·10¹⁰ CFU/g, at least about 7·10¹⁰ CFU/g, at least about 8·10¹⁰ CFU/g, at least about 9·10¹⁰ CFU/g, or at least about 1·10¹¹ CFU/g, or at least about 2·10¹¹ CFU/g, at least about 3·10¹¹ CFU/g, at least about 4·10¹¹ CFU/g, at least about 5·10¹¹ CFU/g, at least about 6·10¹¹ CFU/g, at least about 7·10¹¹ CFU/g, at least about 8·10¹¹ CFU/g, at least about 9·10¹¹ CFU/g, or at least about 1·10¹² CFU/g.

As used herein, an “effective amount” or an “amount effective for” is defined as an amount effective, at dosages and for periods of time necessary, to achieve a desired biological result, such as reducing, preventing, or treating a disease or condition and/or inducing a particular beneficial effect. The effective amount of compositions of the disclosure may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

As a result of developmental immaturity, exposure to supplemental oxygen, and gut and lung dysbiosis, preterm infants are especially vulnerable to respiratory and gut morbidity. Early exposure to harmful stimuli increases the risk for poor long-term outcomes. In a population of infants that developed bronchopulmonary dysplasia (BPD), consistent decreases in airway microbiome population diversity were observed. Infants predisposed to developing BPD showed a decreased abundance of Lactobacilli in their airways. As in the lungs, gut dysbiosis can trigger pro-inflammatory pathways and exacerbate poor nutrient uptake. Postnatal growth restriction induces an increase in intestinal proteobacteria in BPD and pulmonary hypertension (Wedgwood S, Gerard K, Halloran K, Hanhauser A, Monacelli S, Warford C, Thai P N, Chiamvimonvat N, Lakshminrusimha S, Steinhorn R H, Underwood M A (2020) Intestinal Dysbiosis and the Developing Lung: The Role of Toll-Like Receptor 4 in the Gut-Lung Axis. Front Immunol 11:357. doi:10.3389/fimmu.2020.00357). Meta-analyses of probiotic supplementation trials suggest that multi-strain formulations may reduce necrotizing enterocolitis (NEC) and all-cause mortality (Poindexter B (2021) Use of Probiotics in Preterm Infants. Pediatrics 147 (6). doi:10.1542/peds.2021-051485). Furthermore, the gut and lung microbiome (gut-lung axis) appear to be connected and imbalance in either may lead to abnormal inflammatory responses.

Airway dysbiosis in infants suffering from BPD is characterized by a high abundance of proteobacteria and low abundance of Lactobacilli (Lal C V, Travers C, Aghai Z H, Eipers P, Jilling T, Halloran B, Carlo W A, Keeley J, Rezonzew G, Kumar R, Morrow C, Bhandari V, Ambalavanan N (2016) The Airway Microbiome at Birth. Scientific Reports 6 (1):31023. doi:10.1038/srep31023). The three strains of Lactobacillus included in the formulation have shown to be effective in reducing markers of neutrophilic inflammation in the lung epithelium in vitro and in vivo (US 2021/0361726 A1). Bifidobacteria administration affects the gut microbiome composition of low-birth-weight infants, with a 3-strain blend of B. breve, B. infantis, and B. longum keeping detectable levels low of Clostridium and Enterobactericeae (Ishizeki S, Sugita M, Takata M, Yaeshima T (2013) Effect of administration of bifidobacteria on intestinal microbiota in low-birth-weight infants and transition of administered bifidobacteria: a comparison between one-species and three-species administration. Anaerobe 23:38-44. doi:10.1016/j.anaerobe.2013.08.002). Use of B. lactis and B. infantis in a blend with S. thermophilus in very preterm infants reduced NEC stage 2 or higher (Jacobs S E, Tobin J M, Opie G F, Donath S, Tabrizi S N, Pirotta M, Morley C J, Garland S M (2013) Probiotic effects on late-onset sepsis in very preterm infants: a randomized controlled trial. Pediatrics 132 (6):1055-1062). The composition includes a blend of these 3 anti-inflammatory Lactobacillus strains selected for their respiratory benefits and these 4 Bifidobacterium strains selected for their safety and efficacy in infant gut health. Benefits extend as infants age, with children benefiting from Lactobacilli and Bifidobacteria-based probiotics to decrease URTI symptoms, decrease risk of diarrhea after antibiotic use, and more (Damholt A, Keller M K, Baranowski K, Brown B, Wichmann A, Melsaether C, Eskesen D, Westphal V, Arltoft D, Habicht A, Gao Q, Crawford G (2022) Lacticaseibacillus rhamnosus GG DSM 33156 effects on pathogen defence in the upper respiratory tract: a randomised, double-blind, placebo-controlled paediatric trial. Benef Microbes 13 (1):13-23. doi:10.3920/bm2021.0065; Lukasik J, Dierikx T, Besseling-van der Vaart I, de Meij T, Szajewska H (2022) Multispecies Probiotic for the Prevention of Antibiotic-Associated Diarrhea in Children: A Randomized Clinical Trial. JAMA Pediatr. doi:10.1001/jamapediatrics.2022.1973). Many extremely preterm infants have low Vitamin D levels at birth. From infancy

through childhood, Vitamin D is a key nutrient. Vitamin D3′s immunoregulatory roles and inverse relationship with respiratory illness make it a key nutrient in the mitigation of inflammatory bronchopulmonary disease. Vitamin D3 shows immunomodulatory effects by downregulating NF-kB and reducing IL-12 production (D'Ambrosio D, Cippitelli M, Cocciolo M G, Mazzeo D, Di Lucia P, Lang R, Sinigaglia F, Panina-Bordignon P (1998) Inhibition of IL-12 production by 1,25-dihydroxyvitamin D3. Involvement of NF-kappaB downregulation in transcriptional repression of the p40 gene. J Clin Invest 101 (1): 252-262. doi:10.1172/JCI1050). Vitamin D3 may also play a role in increasing circulating Tregs cells in both healthy and immune-compromised individuals (Fisher S A, Rahimzadeh M, Brierley C, Gration B, Doree C, Kimber C E, Plaza Cajide A, Lamikanra A A, Roberts D J (2019) The role of vitamin D in increasing circulating T regulatory cell numbers and modulating T regulatory cell phenotypes in patients with inflammatory disease or in healthy volunteers: A systematic review. PLOS One 14 (9):e0222313. doi:10.1371/journal.pone.0222313). A phase II trial of early vitamin D supplementation demonstrates a need for vitamin D and sets a safe dosage range (Fort P, Salas A A, Nicola T, Craig C M, Carlo W A, Ambalavanan N (2016) A Comparison of 3 Vitamin D Dosing Regimens in Extremely Preterm Infants: A Randomized Controlled Trial. J Pediatr 174:132-138.e131. doi:10.1016/j.jpeds.2016.03.028).

Lactoferrin is another nutrient that is positively associated with supporting infant health. Lactoferrin is a protein typically found in mammalian milk and has a host of immunological, antibacterial, and antiviral properties (Kell D B, Heyden E L, Pretorius E (2020) The Biology of Lactoferrin, an Iron-Binding Protein That Can Help Defend Against Viruses and Bacteria. Frontiers in Immunology 11 (1221). doi:10.3389/fimmu.2020.01221). Meta-analyses of randomized trials reveal that bovine lactoferrin decreases the risk of late-onset sepsis (LOS) in very preterm infants (Pammi M, Suresh G (2017) Enteral lactoferrin supplementation for prevention of sepsis and necrotizing enterocolitis in preterm infants. Cochrane Database Syst Rev 6 (6):Cd007137. doi:10.1002/14651858.CD007137.pub5; Doyle L W, Cheong J L Y (2019) Does bovine lactoferrin prevent late-onset neonatal sepsis? Lancet 393 (10170):382-384. doi:10.1016/s0140-6736(18)32390-0). Additionally, it can modulate gut permeability and has anti- infective properties (Manzoni P (2016) Clinical Benefits of Lactoferrin for Infants and Children. The Journal of Pediatrics 173: S43-S52. doi:10.1016/j.jpeds.2016.02.075).

Zinc is an important nutrient for growth, cell differentiation, and immune function, especially in preterm infants undergoing a period of rapid postnatal growth. Randomized trials also suggest that zinc supplementation improves growth outcomes in preterm infants (Lassi Z S, Kurji J, Oliveira C S, Moin A, Bhutta Z A (2020) Zinc supplementation for the promotion of growth and prevention of infections in infants less than six months of age. Cochrane Database Syst Rev 4 (4):Cd010205. doi:10.1002/14651858.CD010205.pub2). Supplementation with zinc is associated with an improvement in weight gain and linear growth in preterm neonates (Staub E, Evers K, Askie L M (2021) Enteral zinc supplementation for prevention of morbidity and mortality in preterm neonates. Cochrane Database Syst Rev 3 (3):Cd012797. doi:10.1002/14651858.CD012797.pub2).

As used herein, the term “zinc” is understood to be a zinc salt, a nutrient which is provided in ionic form, such as, for example, zinc gluconate which includes a Zn²⁺ cation. Other zinc salts are contemplated including, but not limited to, zinc oxide, zinc acetate, zinc citrate, and zinc glycinate.

A variety of nutrients may confer additional health benefits for infants and children. DHA and ARA are long chain polyunsaturated fatty acids that may reduce the incidence of respiratory illness and diarrhea in children (Lapillonne A, Pastor N, Zhuang W, Scalabrin D M (2014) Infants fed formula with added long chain polyunsaturated fatty acids have reduced incidence of respiratory illnesses and diarrhea during the first year of life. BMC Pediatr 14:168. doi:10.1186/1471-2431-14-168). Prebiotic fiber, commonly in the form of inulin, can improve stool consistency, reduce incidence of infectious events, and increase Bifidobacterium and Lactobacillus growth in the gut (Lohner S, Jakobik V, Mihályi K, Soldi S, Vasileiadis S, Theis S, Sailer M, Sieland C, Berényi K, Boehm G, Decsi T (2018) Inulin-Type Fructan Supplementation of 3-to 6-Year-Old Children Is Associated with Higher Fecal Bifidobacterium Concentrations and Fewer Febrile Episodes Requiring Medical Attention. The Journal of Nutrition 148 (8):1300-1308. doi:10.1093/jn/nxyl20; Knol J, Scholtens P, Kafka C, Steenbakkers J, Gro S, Helm K, Klarczyk M, Schöpfer H, Böckler H M, Wells J (2005) Colon microflora in infants fed formula with galacto- and fructo-oligosaccharides: more like breast-fed infants. J Pediatr Gastroenterol Nutr 40 (1):36-42. doi:10.1097/00005176-200501000-00007).

Furthermore, human milk oligosaccharides (HMOs) may be added as a nutritional ingredient. Useful HMOs may include, but are not limited to, 2′-Fucosyllactose (2′-FL), lacto-N-tetraose (LNT), lacto-N-neotetraose (LN(n)T), Difucosyl-lactose (DFL), 6′-Sialyllactose (6SL), and 3′-Sialyllactose (3SL), among other similar compounds.

This probiotic and micronutrient blend for infant and children's health is unique in that it combines a spectrum of commensal bacteria with nutrients targeting common deficiencies of prematurity. Other products are meant to provide comprehensive nutrition in one infant formula; however, our product is meant only as a supplement. Administration methods may be orally dosed directly, mixed in formula or breast milk for infants, or delivered in a chewable for older children. Our composition serves to seed the young intestinal microbiome with beneficial bacteria known to improve development outcomes and reduce pulmonary inflammation while ameliorating common nutrient deficiencies.

The method described herein effects maintenance of healthy gut microflora in an individual, such as a human infant or child.

In certain embodiments, the compositions comprising one or more of Lactobacillus (L. plantarum (RSB11™), L. acidophilus (RSB12™), and L. rhamnosus (RSB13™)) and Bifidobacterium (B. lactis (RSB14™), B. infantis (RSB15™), B. breve (RSB16™), or B. longum (RSB17™) can include one or more dry carriers selected from the group consisting of trehalose, maltodextrin, rice flour, microcrystalline cellulose, magnesium stearate, inositol, fructooligosaccharide, galactooligosaccharide, dextrose, and the like. In certain embodiments, the dry carrier can be added to the compositions comprising one or more of the above bacterial strains in a weight percentage of from about 1% to about 95% by weight of the composition.

In certain embodiments, the compositions comprising one or more of the above bacterial strains can include one or more liquid or gel-based carriers, selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents, all compatible with the organism; thickening agents selected from the group consisting of corn starch, guar gum, xanthan gum, and the like; one or more spore germination inhibitors selected from the group consisting of hyper-saline carriers, methylparaben, guar gum, polysorbate, preservatives, and the like. In certain embodiments, the one or more liquid or gel-based carrier(s) can be added to the compositions comprising one or more of the above bacterial strains in a weight/volume percentage of from about 0.6% to about 95% weight/volume of the composition. In certain embodiments, the natural or synthetic flavoring(s) can be added to the compositions comprising one or more of the above bacterial strains in a weight/volume percentage of from about 3.0% to about 10.0% weight/volume of the composition. In certain embodiments, the coloring agent(s) can be added to the compositions comprising one or more of the above bacterial strains in a weight/volume percentage of from about 1.0% to about 10.0% weight/volume of the composition. In certain embodiments, the thickening agent(s) can be added to the compositions comprising one or more of the above bacterial strains in a weight/volume percentage of about 2% weight/volume of the composition.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used.

For oral administration, probiotics may be further combined with one or more solid inactive ingredients for the preparation of tablets, capsules, pills, powders, granules, or other suitable dosage forms. For example, the active agent may be combined with at least one excipient selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like.

The components of the compositions administered according to the methods of the present disclosure can be administered in a wide variety of oral dosage forms. It will be obvious to those skilled in the art that the following dosage forms may comprise, in certain embodiments, as the active component, either a chemical compound of the present disclosure or an acceptable salt of a chemical compound of the present disclosure.

For preparing nutraceutical compositions to be administered according to the methods of the present disclosure, nutraceutically acceptable carriers can be either solid or liquid. Solid form preparations include powders, tablets, pills, capsules, and cachets. A solid carrier can be one or more substances that may also act as diluents, flavoring agents, solubilizers, lubricants, suspending agents, binders, preservatives, tablet disintegrating agents, or encapsulating materials.

In powders, the carrier is a finely divided solid, which is in a mixture with the finely divided active component. In tablets, the active component is mixed with the carrier having the necessary binding capacity in suitable proportions and compacted in the shape and size desired.

In certain embodiments, powders and tablets administered according to methods of the present disclosure preferably may contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, tablets, powders, capsules, pills, sachets, and lozenges are included. Tablets, powders, capsules, pills, sachets, and lozenges can be used as solid forms suitable for oral administration.

Liquid preparations include, but are not limited to, solutions, suspensions, and emulsions, for example, water or water-propylene glycol solutions. The compositions may take such forms as suspensions, solutions, or emulsions in oily or aqueous vehicles, and may contain formulation agents such as suspending, stabilizing, and/or dispersing agents. Oil carriers include, but are not limited to, sunflower oil, cranberry seed oil, algal oil, palm oil, coconut oil, and rice bran oil. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth, or buccal, or sublingual administration include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

The nutraceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, sachet, or lozenge itself; or it can be the appropriate number of any of these in packaged form.

Tablets, capsules, and lozenges for oral administration and liquids for oral use are preferred compositions.

Further details on techniques for formulation and administration may be found in the latest edition of REMINGTON'S PHARMACEUTICAL SCIENCES (Mack Publishing Co., Easton, PA).

### Routes of Administration

The compounds may be administered by any route, including, but not limited to, oral, sublingual, buccal, or as an oral spray.

The methods described above may be further understood in connection with the following Examples. In addition, the following non-limiting examples are provided to illustrate the invention. However, the person skilled in the art will appreciate that it may be necessary to vary the procedures for any given embodiment of the invention, e.g., vary the order or steps.

In one embodiment, the addition of micronutrients Vitamin D, lactoferrin, and zinc to a blend of 7 commensal Lactobacillus and Bifidobacterium strains helps support early infant health and development. Additional formulations may include DHA+ARA and prebiotic fiber.

The following nonlimiting combinations of strains and nutrients are contemplated herein.

The invention described herein is embodied in the following non-limiting examples. In the examples below, the term “B CFU” is defined as billion(s) of CFUs.

### EXAMPLE 1

### ResB Blend 2

Table 1 shows one preferred combination of the seven bacterial strains as described herein.

TABLE 1

| | | | B CFU | |
|---|---|---|---|---|
| CFU Basis | | 4 | total | |
| | B | B | Mass | |
| Ingredient | CFU/g | CFU | (mg) | |
| L. plantarum | 400 | 1.143 | 2.86 | 3.19% |
| L. acidophilus | 200 | 1.143 | 5.71 | 6.38% |
| L. rhamnosus | 350 | 1.143 | 3.27 | 3.65% |
| B. lactis | 300 | 1.143 | 3.81 | 4.25% |
| B. infantis | 50 | 1.143 | 22.86 | 25.52% |
| B. breve | 300 | 1.143 | 3.81 | 4.25% |
| B. longum | 100 | 1.143 | 11.43 | 12.76% |
| Dry filler and | | | 35.83 | 40.00% |
| flowability aid | | | | |
| SUM | | | 89.57 | mg |

### EXAMPLE 2

Table 2 shows another preferred combination of pediatric nutrition grade Bifidobacterium and Lactobacillus strains selected from the list of seven as described herein.

TABLE 2

| Strain/Ingredient | B CFU per dose | mg/g |
|---|---|---|
| B. lactis Bi-07 | 27.13 | 42.50 |
| B. lactis DSM 15954 | 26.08 | 42.50 |
| B. lactis HN019 | 26.68 | 31.90 |
| L. acidophilus NCFM | 16.88 | 63.80 |
| L. rhamnosus HN001 | 75.55 | 127.60 |
| L. rhamnosus GG | 15.04 | 36.50 |
| L. plantarum Lp-202195 | 97.62 | 255.23 |
| Rice maltodextrin | | 399.97 |
| SUM | 274.99 billion CFU | 1000 MG/G |

### EXAMPLE 3

### ResB Blend 2 Used in a Dry Powder Sachet—Infant Formulation A

TABLE 3

| Ingredient | B CFU/g | B CFU (label) | Mass (mg) |
|---|---|---|---|
| resB Blend 2 | 4 | 8.0 | 89.6 |
| L. plantarum | 400 | 1.143 | 2.9 |
| L. acidophilus | 200 | 1.143 | 5.7 |
| L. rhamnosus | 350 | 1.143 | 3.3 |
| B. lactis | 300 | 1.143 | 3.8 |
| B. infantis | 50 | 1.143 | 22.9 |
| B. breve | 300 | 1.143 | 3.8 |
| B. longum | 100 | 1.143 | 11.4 |
| Dry filler and flowability aid | | | 35.8 |
| Silica | | | 8.0 |
| FOS (fructooligosaccharide) | | | 500.0 |
| Dry filler and flowability aid | | | 758.6 |
| SUM-Active mass | | | 1445.7 |
| Vitamin D3 (100,000 IU/g) | | | 3.0 |
| Zinc Gluconate | | | 2.0 |
| Lactoferrin | | | 150.0 |
| SUM-extracts | | | 155.0 |
| Sachet fill | | | 1600.7 |

### EXAMPLE 4

### ResB Blend 2 Used in an Oil Drop Formula-Infant Formulation B

TABLE 4

| Ingredient | B CFU/g | B CFU (label) | Mass (mg) |
|---|---|---|---|
| resB Blend 2 | 4 | 8.0 | 89.6 |
| L. plantarum | 400 | 1.143 | 2.9 |
| L. acidophilus | 200 | 1.143 | 5.7 |
| L. rhamnosus | 350 | 1.143 | 3.3 |
| B. lactis | 300 | 1.143 | 3.8 |
| B. infantis | 50 | 1.143 | 22.9 |
| B. breve | 300 | 1.143 | 3.8 |
| B. longum | 100 | 1.143 | 11.4 |
| SUM-Active mass | | | 89.6 |
| Silicon dioxide | | | 1.5 |
| Mixed tocopherol | | | 0.5 |
| Vitamin D3 (100,000 IU/g) | | | 3.0 |
| Zinc Gluconate | | | 1.5 |
| Lactoferrin | | | 150.0 |
| SUM-extracts | | | 154.5 |
| Suspension fill | | | 244.1 |
| Sunflower Oil dose | | | 273 |

EXAMPLE 5

### ResB Blend 2 Used in a Children's Chewable Formulation

TABLE 5
resKid Formulation

| | B | B CFU | Mass |
|---|---|---|---|
| Ingredient | CFU/g | label | (mg) |
| L. plantarum | 400 | 1.143 | 5.7 |
| L. acidophilus | 200 | 1.143 | 11.4 |
| L. rhamnosus | 350 | 1.143 | 6.5 |
| B. lactis | 300 | 1.143 | 7.6 |
| B. infantis | 50 | 1.143 | 45.7 |
| B. breve | 300 | 1.143 | 7.6 |
| B. longum | 100 | 1.143 | 22.9 |
| ACTIVE MASS | | | 107.5 |
| Mannitol (binder) | | | 300.0 |
| Prebiotic fiber | | | 300.0 |
| DHA & ARA blend | | | 375.0 |
| Dry filler and flowability aid | | | 150.0 |
| Vitamin D3 (100,000 IU/g) | | | 3.0 |
| Zinc Gluconate | | | 1.5 |
| Lactoferrin | | | 150.0 |
| Chewable mass | | | 1,387.0 |

### EXAMPLE 6

ResB Blend 2 Used in a Dry Powder Stick Pack—Synbiotic Formulation

TABLE 6
Performance synbiotic

| Ingredient | B CFU/g | B CFU (label) | Mass (mg) |
|---|---|---|---|
| resB Blend 2 | | 30 | |
| L. plantarum | 400 | 4.285714286 | 10.7 |
| L. acidophilus | 200 | 4.285714286 | 21.4 |
| L. rhamnosus | 350 | 4.285714286 | 12.2 |
| B. lactis | 300 | 4.285714286 | 14.3 |
| B. infantis | 50 | 4.285714286 | 85.7 |
| B. breve | 300 | 4.285714286 | 14.3 |
| B. longum | 100 | 4.285714286 | 42.9 |
| ACTIVE MASS | | | 201.5 |
| Prebiotic fiber | | | 5000.0 |
| DHA & ARA blend | | | 600.0 |
| MCC | | | 150.0 |
| Vitamin D3 (100,000 IU/g) | | | 6.0 |
| Zinc Gluconate | | | 3.0 |
| Stick pack mass | | | 5,960.5 |

Various combinations are contemplated as shown in the above Examples. Dosages may be varied as appropriate to age, height, and weight of the human subject.

### Cell Culture and In Vitro Testing

Bacterial inhibition studies. As shown in the Figures, inhibition studies verified that potential micronutrient ingredients do not inhibit the growth of the probiotic strains included in the product.

Stock Preparations of Bifidobacterium strains. The following strains of bacterial species are commercially available.

Each individual bacterial species (B. lactis Bi-07 a.k.a. RSB14TM, B. infantis Bi-26a.k.a. RSB 15TM, B. breve Bb-18 a.k.a. RSB 16™, and B. longum B1-05 a.k.a. RSB17™was grown independently in 0.05% cysteine MRS broth, anaerobically in a rotary incubator for 72 hr at 37° C. Anaero packs were used in the incubator culture vessel to create an anaerobic environment. 72 hr incubation is necessary to reach genus-specific log growth phase prior to taking measurements.

Lactobacillus strains (L. plantarum, L. acidophilus, and L. rhamnosus) were grown independently in an aerobic incubator for 24 hr at 37° C.

Each individual culture tube was measured for cell density by OD600 measurement (n=3) at a 1:10 dilution in 0.05% cysteine MRS Broth. The OD600 values were blank adjusted, and then dilution adjusted.

Supplement stocks (Lactoferrin, Vitamin D3, and Zinc Gluconate) were made in separate 15 mL tubes by weighing out 150 mg of each supplement and putting them in 10 mL of 0.05% cysteine MRS Broth to bring them to a final concentration of 15 mg/mL. 1 mL of each of the supplement stock was then diluted into 9 mL of 0.05% cysteine MRS Broth to create a second stock at a final concentration of 1.5 mg/mL. This was then repeated, using the second stock solutions to create a third stock solution with a final concentration 0.15 mg/mL.

1 mL of each supplement stock solution was then vortexed and aliquoted into 4 separate 1.7 mL tubes, one tube for each bacteria species.

Optical Density was measured at 600 nm (OD600). Each bacterial culture stock was diluted and standardized to a concentration of 1.00E+08 cells/mL. 75 microliters (μL) of each standardized bacteria stock was then vortexed and pipetted into each respective supplement media 1.7 mL tube. Each individual culture tube, and blank supplement media, was measured for cell density by OD600 measurement (n=3) at a 1:10 dilution in the corresponding supplement stock media and recorded. The 1.7 mL tubes were then incubated anaerobically in a rotary incubator for 48 hr at 37° C.

Each individual culture tube was then remeasured for cell density by OD600 measurement (n=3) at a 1:10 dilution in the corresponding supplement stock media and recorded.

### Culture Plating—Colony Forming Units (CFU) Measurement

Each bacterial culture stock was diluted and standardized to a concentration of 1.00E+06 cells/mL.

10 μL of each standardized bacteria stocks was then vortexed and pipetted into each respective supplement media 1.7 mL tube and then incubated anaerobically for 4 hr at 37° C.

50 μL of each growth stock was then plated onto a 0.05% cysteine MRS Agar plate, and then incubated anaerobically for 72 hr at 37 C. The plates were then counted and the CFU per plate was recorded.

The growth of bacterial strains was determined as discussed above.

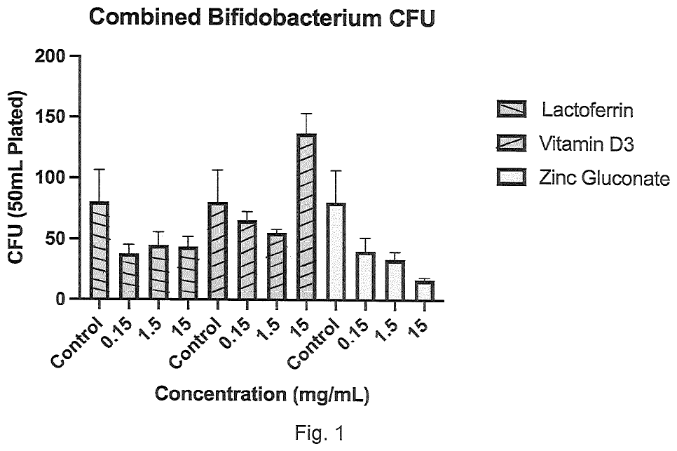

As shown in FIG. 1, the combined bacterial strains B. lactis (RSB14™), B. infantis (RSB15™M), B. breve (RSB16™), and B. longum (RSB 17™) were not inhibited by any of lactoferrin, Vitamin D3, or zinc gluconate when treated with those test compounds at 0.15 mg/mL, 1.5 mg/mL, and 15 mg/mL, respectively.

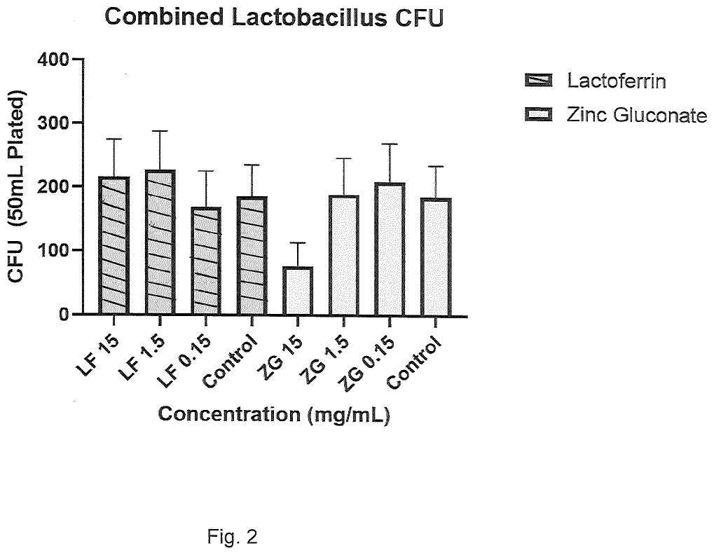

As shown in FIG. 2, the combined bacterial strains L. plantarum (RSB11™), L. acidophilus (RSB12™M), and L. rhamnosus (RSB13™) were not inhibited by any of lactoferrin or zinc gluconate when treated with those test compounds at 0.15 mg/mL, 1.5 mg/mL, and 15 mg/mL, respectively.

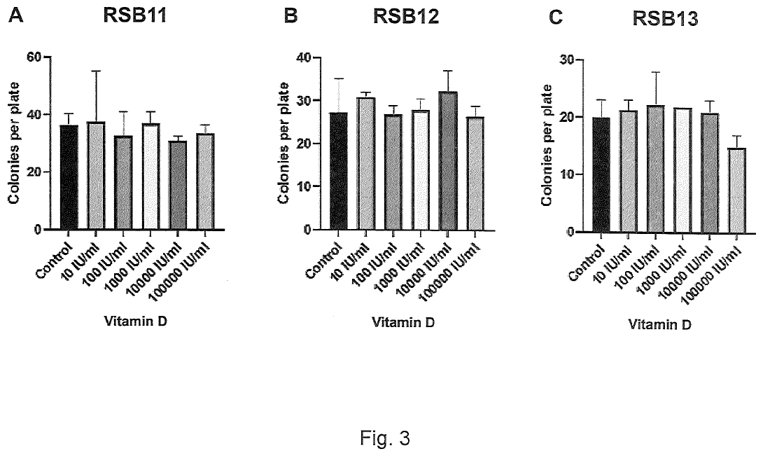

As shown in FIG. 3, the individual strains L. plantarum (RSB11™M), L. acidophilus (RSB12™), and L. rhamnosus (RSB13™) were not inhibited by Vitamin D3 when each was treated with Vitamin D3 at 10 IU/mL, 100 IU/mL, 1,000 IU/mL, 10,000 IU/mL, and 100,000 IU/mL, respectively.

Matrix metalloproteinase 9 (MMP-9) is a class of enzymes that belong to the zinc-metalloproteinases family involved in the degradation of the extracellular matrix. MMP-9 may be upregulated during pathological processes such as in proliferation of E. coli or infections. Therefore, reduction of the MMP-9 marker may be beneficial to an organism, including humans.

In vitro E. coli exposure and treatment with Vitamin D3 or Bifidobacterium strains

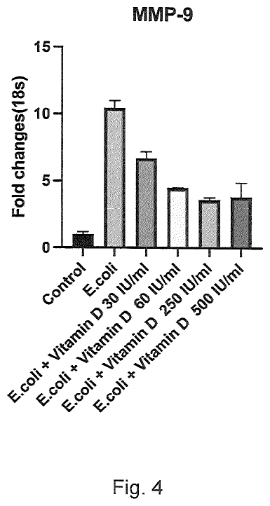

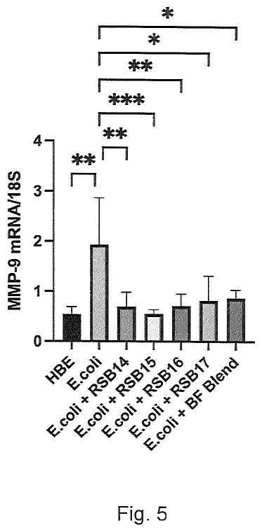

Human bronchial epithelial (HBE) cells were exposed to E. coli (5×10⁷ CFU/mL) and treated with increasing concentrations of Vitamin D3 (FIG. 4) or Bifidobacterium strains (FIG. 5).

As shown in FIG. 4, human bronchial epithelial (HBE) cells were exposed to E. coli with Vitamin D3 at 30 IU/mL, 60 IU/mL, 30 IU/mL, and 30 IU/mL, which reduced MMP-9 mRNA levels (fold changes, 18S RNA).

10 mg/mL of each individual Bifidobacterium strain (B. lactis (RSB14™), B. infantis (RSB15™), B. breve (RSB16™), and B. longum (RSB17™) strains) was cultured in MRS broth for 72 hours as described above. For the BF blend, 2.5 mg/mL of each strain was combined for a total of 10 mg/mL and cultured. Human bronchial epithelial cells (HBE) were exposed to E. coli (5×10⁷ CFU/mL) and treated with individual strains or BF blend. MMP-9 mRNA expression was measured by qPCR.

As shown in FIG. 5, MMP-9 mRNA expression was significantly reduced after treatment with each of the strains individually (B. lactis (RSB14™), B. infantis (RSB15™), B. breve (RSB16™), and B. longum (RSB17™)) and with the BF blend.

It is expected that oral delivery of a dietary supplement, food product, or nutraceutical, comprising a blend of bacterial strains selected from the group consisting of Lactobacillus spp. including L. plantarum, L. acidophilus, and L. rhamnosus, and Bifidobacterium spp. including B. lactis, B. infantis, B. breve, and B. longum, as described herein would be useful in supporting health in an infant or child.

For example, a blend as described above may be used to improve gastrointestinal health (thus providing relief from gas, bloating, constipation, loose stool, diarrhea, or other discomfort), and/or provide improvement in respiratory function.

The use of the terms “a,” “an,” “the,” and similar referents in the context of

describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the ° values may range in value above or below the stated value in a range of approximately ±2%; in other embodiments, the values may range in value above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non- claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof, and, accordingly, reference should be made to the appended claims, rather than to the foregoing specification, as indicating the scope of the invention.

1. A dietary supplement, food product, or nutraceutical for use in supporting health in an infant or child, comprising a blend of bacterial strains selected from the group consisting of Lactobacillus spp. including L. plantarum, L. acidophilus, and L. rhamnosus, and Bifidobacterium spp. including B. lactis, B. infantis, B. breve, and B. longum.

2. The dietary supplement of claim 1, comprising a blend of bacterial strains comprising L. plantarum, L. acidophilus, L. rhamnosus, and B. lactis.

3. The dietary supplement of claim 1, comprising a blend of bacterial strains comprising L. plantarum Lp-202195, L. acidophilus NCFM, L. rhamnosus GG, L. rhamnosus HN001, B. lactis HN019, B. lactis DSM 15954, and B. lactis Bi-07.

4. The dietary supplement of claim 1, comprising a blend of bacterial strains comprising B. lactis Bi-07, B. infantis Bi-26, B. breve Bb-18, and B. longum B1-05.

5. The dietary supplement of claim 1, in which at least one Lactobacillus species is viable and capable of proliferating in the infant or child.

6. The dietary supplement of claim 1, in which at least one Lactobacillus species is not capable of proliferating in the infant or child.

7. The dietary supplement of claim 1, in which at least one Lactobacillus species is heat-killed.

8. The dietary supplement of claim 1, in which the bacterial extract of at least one Lactobacillus species is present.

9. The dietary supplement of claim 1, in which at least one Bifidobacterium species is viable and capable of proliferating in the infant or child.

10. The dietary supplement of claim 1, in which at least one Bifidobacterium species is not capable of proliferating in the infant or child.

11. The dietary supplement of claim 1, in which at least one Bifidobacterium species is heat-killed.

12. The dietary supplement of claim 1, in which the bacterial extract of at least one Bifidobacterium species is present.

13. The dietary supplement of claim 1 further comprising at least one nutritionally active ingredient.

14. The dietary supplement of claim 13, wherein the at least one nutritionally active ingredient is selected from the group consisting of a vitamin, a micronutrient, a mineral, a prebiotic fiber, a fatty acid, and an amino acid, or any combination thereof.

15. The dietary supplement of claim 13, wherein the at least one nutritionally active ingredient is selected from the group consisting of vitamin D3, lactoferrin, and zinc gluconate.

16. The dietary supplement of claim 15, further comprising at least one human milk oligosaccharide.

17. The dietary supplement of claim 1 formulated for oral administration.

18. The dietary supplement of claim 17 in which the oral formulation is a capsule, microcapsule, tablet, granule, powder, troche, pill, suspension, solution, or syrup.

19. A method for improving gastrointestinal health in an infant or child, comprising the steps of:
providing a dietary supplement including a blend of bacterial strains selected from the group consisting of Lactobacillus spp. including L. plantarum, L. acidophilus, and L. rhamnosus, and Bifidobacterium spp. including B. lactis, B. infantis, B. breve, and B. longum; and
administering the blend to the infant or child by oral administration.

20. The method of claim 19, wherein gastrointestinal health is improved in the infant or child by reducing a symptom selected from gas, bloating, constipation, loose stool, diarrhea, or other discomfort.

21. The method of claim 19, wherein the blend of bacterial strains comprises B. lactis Bi-07, B. infantis Bi-26, B. breve Bb-18, and B. longum B1-05.

22. The method of claim 19, wherein the dietary supplement further includes at least one nutritionally active ingredient selected from the group consisting of vitamin D3, lactoferrin, zinc gluconate, and a human milk oligosaccharide.

23. A method for improving respiratory function in an infant or child, comprising the steps of:
providing a dietary supplement including a blend of bacterial strains selected from the group consisting of Lactobacillus spp. including L. plantarum, L. acidophilus, and L. rhamnosus, and Bifidobacterium spp. including B. lactis, B. infantis, B. breve, and B. longum; and
administering the blend to the infant or child by oral administration.

24. The method of claim 23, wherein respiratory function is improved in the infant or child by treating a condition or reducing a symptom derived from bronchopulmonary dysplasia (BPD), respiratory inflammation.

25. The method of claims claim 19 or 23, wherein the bacterial strains are provided in a daily dosage of from about 1×10⁹ CFUs to about 1×10¹⁰ CFUs.

26. The method of claim 23, wherein the bacterial strains are provided in a daily dosage of from about 1×10⁹ CFUs to about 1×10¹⁰ CFUs.
